(19) United States
(12) Patent Application Publication
(10) Pub. No.: US 2024/0139257 A1
(43) Pub. Date: May 2, 2024

(54) METHODS AND COMPOSITIONS FOR ALOPECIA TREATMENT USING FIBROBLASTS AND FIBROBLAST-DERIVED PRODUCTS

(71) Applicant: FIGENE, LLC, Webster, TX (US)

(72) Inventors: Thomas ICHIM, San Diego, CA (US); Pete O'HEERON, Houston, TX (US)

(21) Appl. No.: 18/561,979

(22) PCT Filed: May 25, 2022
(86) PCT No.: PCT/US2022/030805

§ 371 Date: Nov. 17, 2023

Related U.S. Application Data
(60) Provisional application No. 63/193,403, filed on May 26, 2021.

(51) Int. Cl. A61K 35/33; A61K 31/122; A61N 5/06; A61P 17/14; C12N 5/077

(52) U.S. Cl. CPC A61K 35/33; A61K 31/122; A61N 5/0617; A61P 17/14; C12N 5/0656

(57) ABSTRACT

Disclosed herein are methods and compositions for treatment and prevention of alopecia, including alopecia areata. Aspects are directed to methods for stimulating or accelerating hair follicle regeneration. Certain aspects are directed to methods for treating alopecia areata comprising administration of fibroblasts and/or a fibroblast derived product to a subject suffering from alopecia areata. In some cases, a fibroblast derived product is conditioned media, exosomes, or apoptotic bodies from fibroblasts, formulated for topical use for treatment or prevention of alopecia areata.

This application claims priority to U.S. Provisional Patent Application Ser. No. 63/193,403, filed May 26, 2021, which is incorporated by reference herein in its entirety.

### TECHNICAL FIELD

Embodiments of the disclosure encompass at least the fields of cell biology and regenerative medicine.

### BACKGROUND

Hair loss, also known as alopecia or baldness, refers to a loss of hair from part of the head or body. The severity of hair loss can vary from a small area to the entire body. Alopecia may be caused by psychological distress [1, 2] or may be drug induced [3-7], among other causes. Common types of alopecia include: Androgenetic alopecia (including male-pattern hair loss and female-pattern hair loss), alopecia areata, and a thinning of hair known as telogen effluvium [8-17]. Causes of male-pattern hair loss include a combination of genetics and male hormones, causes of female pattern hair loss are unclear, the cause of alopecia areata is autoimmunity, and the cause of telogen effluvium is typically a physically or psychologically stressful event [18, 19].

The hair follicle is a regenerating organ where stem cells enable a massive large-scale renewal. The hair follicle is composed of an outer root sheath, an inner root sheath, and the hair shaft. The proliferating undifferentiated matrix cells give rise to the inner root sheath and the hair shaft and are surrounded by a dermal papilla of specialized mesenchymal cells. The dermal papilla instructs the formation of the follicle, but the characteristics of the follicle are acquired by epithelial information. The lower portion of the follicle goes through a growth cycle that involves the phases of anagen (active growth), catagen (destruction) and telogen (quiescence). These different phases last for varying time periods depending on the hair follicle location and function. The matrix cells proliferate rapidly during the anagen phase, migrate upwards then differentiate into the cell types of the inner root sheath and hair shaft. During the catagen phase, the lower follicle undergoes apoptotic death and the dermal papilla moves upwards until it reaches the area beneath the bulge. It remains there during telogen. Once the dermal papilla recruits stem cells from the bulge, anagen begins anew and the follicle can regenerate through proliferation and differentiation.

The management of alopecia generally involves use of medication or surgery [20]. Medicines currently used in treatment of alopecia are minoxidil, finasteride, and dutasteride [21-23]. The management of alopecia by surgery involves hair transplantation. Hair transplantation is usually carried out under local anaesthesia. A surgeon will move healthy hair from the back and sides of the head to areas of thinning. The procedure can take between four and eight hours, and additional sessions can be carried out to make hair even thicker. Conventional hair transplantation process suffers when there are not enough hairs in the non-bald areas. Stem cell therapy represents an emerging strategy for treatment and management of alopecia [24]. However, in the context of conventional hair transplantation, administration of hair follicle stem cells into the bald scalp zone has a very low efficiency since there are no required growth factors for the growth of new follicles in the skin of such a zone.

There exists a need for additional and improved methods and compositions for treatment of alopecia, including androgenetic alopecia and alopecia areata.

### BRIEF SUMMARY

Aspects of the present disclosure are directed to fibroblasts and fibroblast-derived products, and methods of use thereof in treatment and prevention of alopecia. Certain embodiments are directed to methods for treatment of alopecia, including androgenetic alopecia and alopecia areata, comprising providing fibroblasts or fibroblast-derived products to a subject having alopecia. Also disclosed are methods for prevention of alopecia, including androgenetic alopecia and alopecia areata, comprising providing fibroblasts or fibroblast-derived products to a subject at risk for developing alopecia. Fibroblasts of the disclosure may be subjected to conditions sufficient to enhance regenerative and/or angiogenic activity. Pharmaceutical compositions comprising fibroblasts or fibroblast-derived products for use in treatment or prevention of alopecia are also contemplated herein, including compositions formulated for topical administration such as soaps, shampoos, and ointments. Accordingly, embodiments of the disclosure provide new strategies to treat and prevent alopecia, including androgenetic alopecia and alopecia areata.

Embodiments of the present disclosure include methods for treatment of alopecia, methods for prevention of alopecia, methods for slowing hair loss, methods for stimulating hair growth, and methods for reducing inflammation. Methods of the disclosure may comprise one or more of: diagnosing a subject for alopecia, diagnosing a subject for androgenetic alopecia, diagnosing a subject for alopecia areata, diagnosing a subject for dermal inflammation, providing fibroblasts to a subject, providing fibroblast-derived products to a subject, and providing fibroblast-derived exosomes to a subject. Also disclosed are compositions comprising one or more of: fibroblasts, dermal fibroblasts, fibroblast-derived products, conditioned media from fibroblasts, and fibroblast-derived exosomes. Any one or more of the preceding steps or components may be excluded from certain embodiments of the disclosure.

Disclosed herein, in some embodiments, is a method of treating or preventing alopecia in a subject comprising providing to the subject an effective amount of fibroblasts or fibroblast-derived products. In some embodiments, the alopecia is associated with dermal inflammation. In some embodiments, the alopecia is androgenetic alopecia, male-pattern hair loss, female-pattern hair loss, alopecia areata, or telogen effluvium. In some embodiments, the alopecia is androgenetic alopecia. In some embodiments, the alopecia is alopecia areata. In some embodiments, the fibroblasts or fibroblast-derived products are administered topically. In some embodiments, the fibroblasts or fibroblast-derived products are administered intradermaly and/or transdermally. In some embodiments, the fibroblasts or fibroblast-derived products are administered to a region of the scalp of the subject. In some embodiments, the method further comprises administering a regenerative light source to the region of the scalp of the subject. In some embodiments, the regenerative light source is a laser light.

In some embodiments, the method comprises providing to the subject an effective amount of fibroblasts. In some embodiments, the fibroblasts are allogeneic, xenogeneic, or autologous to the subject. In some embodiments, the fibroblasts are derived from skin, adipose, bone marrow, omental tissue, blood, deciduous teeth, fallopian tubes, testicular tissue, ovarian tissue, hair follicle, endometrial tissue, or a combination thereof. In some embodiments, the fibroblasts are dermal fibroblasts. In some embodiments, the fibroblasts were previously subjected to conditions sufficient to enhance regenerative activity. In some embodiments, the conditions are sufficient to upregulate HIF1a expression in the fibroblasts. In some embodiments, the conditions are sufficient to upregulate HIF1a by at least 25% relative to an untreated control. In some embodiments, the conditions are sufficient to enhance nuclear translocation of HIF1a in the fibroblasts. In some embodiments, the conditions comprise agents capable of imitating hypoxia. In some embodiments, the conditions comprise culturing the fibroblasts with carbon monoxide. In some embodiments, the conditions comprise exposing the fibroblasts to a gas composition comprising between about 0% and about 79% nitrogen by weight, between about 21% and about 99.999999% oxygen by weight, and between 0.0000001% and about 0.3% carbon monoxide by weight. In some embodiments, the gas composition comprises 0% nitrogen and about 99.999999% oxygen. In some embodiments, the gas composition comprises between about 0.005% and about 0.05%. In some embodiments, the fibroblasts were previously subjected to conditions sufficient to enhance survival and/or activity of the fibroblasts. In some embodiments, the conditions comprise treatment with an epigenetic modulator. In some embodiments, the epigenetic modulator is a histone deacetylase inhibitor. In some embodiments, the histone deacetylase inhibitor is valproic acid, vorinostat, entinostat, panobinostat, trichostatin A, mocetinostat, belinostat, FK228, MC1568, tubastatin, sodium butyrate, or sulforaphane. In some embodiments, the epigenetic modulator is a DNA methyltransferase inhibitor. In some embodiments, the DNA methyltransferase inhibitor is 5-azacytidine. In some embodiments, the conditions comprise culturing the fibroblasts with GSK-3 inhibitor. In some embodiments, the GSK-3 inhibitor is lithium or a lithium salt.

In some embodiments, the method comprises providing to the subject an effective amount of fibroblast-derived products. In some embodiments, the fibroblast-derived products were obtained from fibroblasts derived from skin, adipose, bone marrow, omental tissue, blood, deciduous teeth, fallopian tubes, testicular tissue, ovarian tissue, hair follicle, endometrial tissue, or a combination thereof. In some embodiments, the fibroblast-derived products were obtained from dermal fibroblasts. In some embodiments, the fibroblast-derived products were obtained from fibroblasts that are allogeneic, xenogeneic, or autologous to the subject. In some embodiments, the fibroblast-derived products comprise conditioned media from culture of fibroblasts. In some embodiments, the conditioned media was obtained from culture of the fibroblasts in EMEM, alpha-MEM, IMDM, DMEM, or RPMI. In some embodiments, the conditioned media was generated by culture of adherent fibroblasts in a liquid suspension comprising nutrition for the fibroblasts. In some embodiments, the liquid suspension comprises a growth factor. In some embodiments, the liquid suspension comprises stem cell exosomes. In some embodiments, the stem cell exosomes are exosomes from mesenchymal stem cells. In some embodiments, the mesenchymal stem cells were derived from umbilical cord, bone marrow, skin, fallopian tube, adipose tissue, endometrial tissue, peripheral blood, menstrual blood, hair follicle, or a combination thereof. In some embodiments, the liquid suspension comprises a neutralizing factor capable of inhibiting activity of one or more inflammatory mediators. In some embodiments, the neutralizing factor is a monoclonal antibody, an antisense oligonucleotide, or a gene editing system. In some embodiments, the neutralizing factor is an antibody capable of binding to interleukin-1, interleukin-6, interleukin-8, interleukin-9, interleukin-11, interleukin-12, interleukin-15, interleukin-17, interleukin-18, interleukin-21, interleukin-23, interleukin-27, interleukin-33, TNFα, interferon gamma, TNFβ, or lymphotoxin. In some embodiments, the liquid suspension comprises VEGF, EGF, PGDF-BB, IGF-1, HGF-1, NGF, BDNF, IL-3, IL-4, IL-10, IL-13, IL-20, IL-35. In some embodiments, the fibroblast-derived products are microvesicles from fibroblasts. In some embodiments, the fibroblast-derived products are exosomes from fibroblasts. In some embodiments, the exosomes were concentrated from conditioned media from the fibroblasts. In some embodiments, the exosomes were concentrated by a) functionalizing a support with a single stranded oligonucleotide to generate a functionalized support; b) incubating the functionalized support with a ligand having a tag which is complimentary to the single strand oligonucleotide order to obtain an immobilized ligand; c) incubating the immobilized ligand with the conditioned media to allow the capture of the exosomes through the binding of said immobilized ligand with the exosomes to obtain a substrate of captured exosomes; and d) incubating the captured exosomes with a restriction enzyme. In some embodiments, the ligand is an antibody, a peptide, or an aptamer. In some embodiments, the support is a magnetic bead, a membrane, a cell culture plate, a test tube, a slide, a microplate, a microchannel, a pillar, or a disk-like piece. In some embodiments, the support is functionalized with the ligand by covalent bonding or via biotinylation. In some embodiments, the restriction enzyme is a DNAse. In some embodiments, the ligand is an antibody capable of binding to an exosome-specific tetraspanin. In some embodiments, the ligand is an antibody capable of binding to MHC class I and II, HSP70, Annexin V, Flotillin, or EpCAM. In some embodiments, the conditioned media is derived from culture of the fibroblasts in EMEM, alpha-MEM, IMDM, DMEM, or RPMI. In some embodiments, the fibroblast-derived products are apoptotic vesicles from fibroblasts. In some embodiments, the fibroblast-derived products are nucleic acids from fibroblasts. In some embodiments, the fibroblast-derived products were obtained from fibroblasts subjected to conditions sufficient to enhance regenerative activity. In some embodiments, the conditions are sufficient to upregulate HIF1α expression in the fibroblasts. In some embodiments, the conditions are sufficient to upregulate HIF1α by at least 25% relative to an untreated control. In some embodiments, the conditions are sufficient to enhance nuclear translocation of HIF1α in the fibroblasts. In some embodiments, the conditions comprise agents capable of imitating hypoxia. In some embodiments, the conditions comprise culturing the fibroblasts with carbon monoxide. In some embodiments, the conditions comprise exposing the fibroblasts to a gas composition comprising between about 0% and about 79% nitrogen by weight, between about 21% and about 99.999999% oxygen by weight, and between 0.0000001% and about 0.3% carbon monoxide by weight. In some embodiments, the gas composition comprises 0% nitrogen and about 99.999999% oxygen. In some embodiments, the gas composition comprises between about 0.005% and about 0.05%. In some embodiments, the fibroblast-derived products were obtained from fibroblasts subjected to conditions sufficient to enhance survival and/or activity of the fibroblasts. In some embodiments, the conditions comprise treatment with an epigenetic modulator. In some embodiments, the epigenetic modulator is a histone deacetylase inhibitor. In some embodiments, the histone deacetylase inhibitor is valproic acid, vorinostat, entinostat, panobinostat, trichostatin A, mocetinostat, belinostat, FK228, MC1568, tubastatin, sodium butyrate, or sulforaphane. In some embodiments, the epigenetic modulator is a DNA methyltransferase inhibitor. In some embodiments, the DNA methyltransferase inhibitor is 5-azacytidine. In some embodiments, the conditions comprise culturing the fibroblasts with GSK-3 inhibitor. In some embodiments, the GSK-3 inhibitor is lithium or a lithium salt.

In some embodiments, the method further comprises providing to the subject an effective amount of diphenylcyclopropenone. In some embodiments, the method further comprises providing to the subject an effective amount of a c-Met activator (e.g., HGF). In some embodiments, the method further comprises providing to the subject one or more agents capable of stimulating HGF production.

In some embodiments, the subject has a reduced number of cells expressing FoxP3 compared with an age matched control subject. In some embodiments, the subject has a reduced number of cells expressing interleukin-10 compared with an age matched control subject. In some embodiments, the subject has a reduced number of cells expressing interleukin-4 compared with an age matched control subject. In some embodiments, the subject has a reduced number of cells expressing interleukin-13 compared with an age matched control subject. In some embodiments, the subject has a reduced number of cells expressing interleukin-35 compared with an age matched control subject. In some embodiments, the subject has a reduced number of T regulatory cells compared with an age matched control subject. In some embodiments, the subject has a reduced number of myeloid suppressor cells compared with an age matched control subject. In some embodiments, the subject has a reduced number of B cells expressing TIM-1 compared with an age matched control subject. In some embodiments, the subject has a reduced number of B cells expressing IL-10 compared with an age matched control subject. In some embodiments, the subject has a reduced number of B regulatory cells compared with an age matched control subject. In some embodiments, the subject has an increased number of cells expressing interferon gamma compared with an age matched control subject. In some embodiments, the subject has an increased number of cells expressing TNF-alpha compared with an age matched control subject. In some embodiments, the subject has an increased number of cells expressing interleukin-1 compared with an age matched control subject. In some embodiments, the subject has an increased number of cells expressing interleukin-2 compared with an age matched control subject. In some embodiments, the subject has an increased number of cells expressing interleukin-6 compared with an age matched control subject. In some embodiments, the subject has an increased number of cells expressing interleukin-8 compared with an age matched control subject. In some embodiments, the subject has an increased number of cells expressing interleukin-11 compared with an age matched control subject. In some embodiments, the subject has an increased number of cells expressing interleukin-12 compared with an age matched control subject. In some embodiments, the subject has an increased number of cells expressing interleukin-15 compared with an age matched control subject. In some embodiments, the subject has an increased number of cells expressing interleukin-17 compared with an age matched control subject. In some embodiments, the subject has an increased number of cells expressing interleukin-18 compared with an age matched control subject. In some embodiments, the subject has an increased number of cells expressing interleukin-21 compared with an age matched control subject. In some embodiments, the subject has an increased number of cells expressing interleukin-23 compared with an age matched control subject. In some embodiments, the subject has an increased number of cells expressing interleukin-27 compared with an age matched control subject. In some embodiments, the subject has an increased number of cells expressing interleukin-33 compared with an age matched control subject. In some embodiments, the subject has an increased number of natural killer cells compared with an age matched control subject. In some embodiments, the subject has an increased number of natural killer T cells compared with an age matched control subject. In some embodiments, the subject has an increased number of Th1 cells compared with an age matched control subject. In some embodiments, the subject has an increased number of Th17 cells compared with an age matched control subject.

Also disclosed herein, in some embodiments, is a method of treating or preventing androgenetic alopecia in a subject comprising providing to the subject an effective amount of fibroblast-derived exosomes. In some embodiments, disclosed is a method of treating or preventing androgenetic alopecia in a subject comprising providing to the subject an effective amount of conditioned media from fibroblasts.

Also disclosed herein, in some embodiments, is a method of treating or preventing alopecia areata in a subject comprising providing to the subject an effective amount of fibroblast-derived exosomes. In some embodiments, disclosed is a method of treating or preventing alopecia areata in a subject comprising providing to the subject an effective amount of conditioned media from fibroblasts.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, and Claims.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (e.g., the number of population doublings) during the period between passaging depends on many factors, including but not limited to seeding density, substrate, medium, growth conditions, and time between passaging.

As used herein, “conditioned media” describes media in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned media. Conditioned media “from” a cell or population of cells describes conditioned media obtained from the cell or population of cells as described above. Thus, in one example, conditioned media from fibroblasts describes media in which fibroblasts have been cultured for a period of time, and then removed, thus separating the medium from the fibroblasts.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” or “patient” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product” or “fibroblast derivative”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

“Carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### II. Methods and Compositions Treatment or Prevention of Alopecia

Aspects of the present disclosure are directed to methods and compositions for treatment or prevention of alopecia. Alopecia (also “hair loss”) describes any loss of hair from any part of the head of body of a subject. Various types of alopecia are recognized in the art and contemplated herein including, but not limited to, androgenetic alopecia (e.g., male-pattern hair loss, female-pattern hair loss), alopecia areata, and telogen effluvium. In some embodiments, disclosed are methods for treatment of alopecia areata (AA).

As disclosed herein, fibroblasts and/or fibroblast-derived products (e.g., exosomes, conditioned media, etc.) are useful in the treatment and prevention of alopecia. Accordingly, embodiments of the disclosure are directed to methods for treatment or prevention of alopecia comprising providing to a subject an effective amount of fibroblasts or fibroblast-derived products. In some embodiments, methods of the disclosure comprise administration of fibroblasts to a subject. In some embodiments, methods of the disclosure comprise administration of fibroblast-derived products to a subject. In particular embodiments, disclosed are methods comprising administering fibroblast-derived exosomes, or compositions comprising fibroblast-derived exosomes, to a subject for treatment or prevention of alopecia. Administration of such compositions include, for example, topical administration, transdermal administration, and intradermal administration. Contemplated are pharmaceutical compositions comprising fibroblasts or fibroblast-derived products, including, for example, soaps, shampoos, ointments, and other such formulations.

Fibroblasts or fibroblast-derived products may be provided to a subject in combination with one or more therapeutics or therapies. In one example, fibroblasts or fibroblast-derived products of the disclosure are administered to a subject in combination with (e.g., simultaneous with, prior to, or subsequent to) diphenylcyclopropenone. In another example, fibroblasts or fibroblast-derived products of the disclosure are administered to a subject in combination with (e.g., simultaneous with, prior to, or subsequent to) a stimulator of HGF production.

Fibroblasts of the disclosure, including fibroblasts administered to a subject and fibroblasts cultured to obtain fibroblast-derived products, may be subjected to conditions sufficient to enhance regenerative activity of the fibroblasts. In one example, fibroblasts are subjected to such conditions prior to administration to a subject. In another example, fibroblasts are subjected to such conditions prior to obtaining fibroblasts-derived products from the fibroblasts. Such conditions include, for example, conditions sufficient to upregulate HIF1α expression in the fibroblasts, relative to control, untreated fibroblasts. In some embodiments, fibroblasts are subjected to conditions sufficient to upregulate HIF1α expression by at least, at most, or about 10%, 15%, 20%, 25%, 30%, 35%, 40%, 45%, 50%, 55%, 60%, 65%, 70%, 75%, 80%, 85%, 90%, 95%, or 100%, or more, or any range or value derivable therein. Such conditions may be sufficient to enhance nuclear translocation of HIF1α in the cells (i.e., increase the amount of HIF1α in the nucleus of the cells) relative to control, untreated fibroblasts. Such conditions may comprise hypoxic conditions and/or treatment with one or more agents for simulating hypoxia. In some embodiments, fibroblasts of the disclosure are cultured with carbon monoxide. In some embodiments, culturing fibroblasts with carbon monoxide comprises exposing the fibroblasts to a gas composition comprising between about 0% and about 79% nitrogen by weight (or any range or value therein), between about 21% and about 99.999999% oxygen by weight (or any range or value therein), and between 0.0000001% and about 0.3% carbon monoxide by weight (or any range or value therein).

Fibroblasts of the disclosure, including fibroblasts administered to a subject and fibroblasts cultured to obtain fibroblast-derived products, may be subjected to conditions enhance survival and/or activity of the fibroblasts. In one example, fibroblasts are subjected to such conditions prior to administration to a subject. In another example, fibroblasts are subjected to such conditions prior to obtaining fibroblasts-derived products from the fibroblasts. In some embodiments, such conditions comprise treatment with an epigenetic modifier. In some embodiments, the epigenetic modifier is a histone deacetylase inhibitor, such as, for example, valproic acid, vorinostat, entinostat, panobinostat, trichostatin A, mocetinostat, belinostat, FK228, MC1568, tubastatin, sodium butyrate, or sulforaphane. In some embodiments, the epigenetic modifier is a DNA methyltransferase inhibitor such as, for example, 5-azacytidine. In some embodiments, such conditions comprise treatment with a glycogen synthase kinase-3 (GSK-3) inhibitor, such as a GSK-3 beta inhibitor. In some embodiments, the GSK-3 inhibitor is lithium or a lithium salt.

Embodiments of the present disclosure include utilization of fibroblasts and/or fibroblast-derived products (e.g., exosomes) to reduce T cell infiltration into the proximity of the hair bulb in patients with AA. It is known in AA patients that topical immunotherapy with diphenylcyclopropenone (DCP) possesses some therapeutic benefits [25-41]. Using semiquantitative reverse transcription-polymerase chain reaction with RNA extracted from scalp biopsies from patients with AA before and after successful treatment with DCP, and from healthy controls scientists detected a T-cell response with increased steady state mRNA levels for interferon (IFN)-gamma, interleukin (IL)-1 beta, and IL-2 in untreated AA of the total type. After DCP treatment, the IFN-gamma expression was reduced but still above the constitutive level found in controls, whereas mRNA expression of IL-2, IL-8, IL-10, and tumor necrosis factor-alpha was increased. These results point towards cytokines involved in the pathogenesis in AA. A TH1 type cytokine pattern is present in untreated AA, and this is modified by cytokines secreted during DCP treatment. IL-10 has recently been described as an immunomodulator of the TH1 response and, therefore, it is hypothesized that basal keratinocytes or lesional T cells secrete bioactive IL-10 after DCP application, resulting in an inhibitory effect on lesional T lymphocytes [42].

In one embodiment of the disclosure, fibroblasts are administered to a subject at a frequency and concentration sufficient to reduce expression of interferon gamma. In another embodiment, fibroblasts and/or fibroblast-derived products (e.g., exosomes) are administered together with DCP to increase efficacy of DCP treatment in increasing hair regeneration/decreasing hair loss. In another embodiment of the invention fibroblast-derived products such as exosomes are administered at a frequency and concentration sufficient to reduce expression of interferon gamma. Certain example methods for generation of exosomes for immune modulatory therapy are recognized in the art and contemplated herein [43].

In one embodiment of the disclosure, oxidative/inflammatory stress is utilized as a marker of tendency towards alopecia in order to guide dosage and/or frequency of fibroblast and/or fibroblast-derived product administration. Means of assessing oxidative/inflammatory stress are described in the art and contemplated herein [44-66]. In some situations the disclosure provides administration of fibroblasts and/or fibroblast-derived products together with antioxidants.

In some embodiments stimulators of HGF (i.e., agents capable of stimulating HGF production) are added to enhance proliferation of hair follicle cells [67-70].

### III. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In some embodiments, disclosed herein are fibroblasts capable of alopecia, including androgenetic alopecia and alopecia areata. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MEW class I, MEW class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF.

Fibroblasts may be expanded and utilized by administration themselves, or may be cultured in a growth media in order to obtain conditioned media. The term Growth Medium generally refers to a medium sufficient for the culturing of fibroblasts. In particular, one medium for the culturing of the cells of the disclosure herein comprises Dulbecco's Modified Essential Media (DMEM). One example is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone™, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.).

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors). The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco). One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors. In some embodiments fibroblasts are used from young donors. In another embodiment fibroblasts are transfected with genes to allow for enhanced growth and overcoming of the Hayflick limit. Subsequent to derivation of cells, cells may be expanded in culture using standard cell culture techniques.

An example procedure for obtaining fibroblasts from a biopsy is provided. Skin tissue (dermis and epidermis layers) may be biopsied from a subject's post-auricular area. In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Heidelberg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37.0±2° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37.0±2° C. with 5.0±1.0% CO₂ and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37.0±2° C. with 5.0±1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest. When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote angiogenesis. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, ILL IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Aspects of the present disclosure comprise generating conditioned media from fibroblasts. Conditioned media may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500×g). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing alopecia, including androgenetic alopecia and alopecia areata. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### IV. Isolation and Purification of Membrane Vesicles

Exosomes of the disclosure may be obtained from fibroblasts (e.g., from conditioned media from fibroblast culture) and purified for use in the disclosed methods and compositions. In some embodiments, exosomes may be purified by anion exchange, in some cases under pressure. In some embodiments, exosomes of the disclosure are purified by high performance liquid chromatographs (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. Therefore, in a specific embodiment, this disclosure relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, it is preferable to use supports in bead form. In some embodiments, these beads have a homogeneous and calibrated diameter, with a sufficiently high porosity to enable the penetration of the objects under chromatography (e.g., the exosomes). In this way, given the diameter of exosomes (generally between 50 and 100 nm), in certain aspects of the disclosure, it is preferable to use high porosity gels, particularly between 10 nm and 5 μm, for example between approximately 20 nm and approximately 2 μm, such as between about 100 nm and about 1 μm. For the anion exchange chromatography, the support used must be functionalized using a group capable of interacting with an anionic molecule. Generally, this group is composed of an amine which may be ternary or quaternary, which defines a weak or strong anion exchanger, respectively. In some embodiments, it is particularly advantageous to use a strong anion exchanger. In this way, according to the disclosure, a chromatography support as described above, functionalized with quaternary amines, is used. Therefore, according to a more specific embodiment, the anion exchange chromatography is performed on a support functionalized with a quaternary amine. In some embodiments, the support is selected from poly(styrene-divinylbenzene), acrylamide, agarose, dextran and silica, alone or in mixtures, and functionalized with a quaternary amine. Examples of supports functionalized with a quaternary amine include the gels SOURCE Q, MONO Q, Q SEPHAROSE®, POROS™ HQ and POROS™ QE, FRACTOGEL™ TMAE type gels and TOYOPEARL SUPER™ Q gels.

A support which may be used to perform anion exchange chromatography includes poly(styrene-divinylbenzene). An example of this type of gel which may be used within the scope of this invention is SOURCE Q gel, for example SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, for example using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μl up to 10 ml or greater. In this way, the supports available have a capacity which may reach 25 mg of proteins/ml, for example. For this reason, a 100 μl column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the disclosure, a gel permeation chromatography step is added to the anion exchange step, either before or after the anion exchange chromatography step. In this embodiment, the permeation chromatography step may take place after the anion exchange step. In a specific embodiment, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX™ 200HR (Pharmacia), TSK G6000 (TosoHaas) or SEPHACRYL™ S (Pharmacia) may be used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles. In this respect, in a specific embodiment of the disclosure, a biological sample is a culture supernatant of membrane vesicle-producing fibroblast cells.

In addition, according to a certain embodiment of the disclosure, the biological sample is treated, prior to the chromatography step, to be enriched with membrane vesicles (enrichment stage). In this way, in a specific embodiment, the present disclosure provides a method of preparing membrane vesicles from a biological sample, characterized in that it comprises at least: 1) an enrichment step, to prepare a sample enriched with membrane vesicles, and 2) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells (e.g., fibroblasts) or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In another specific embodiment, the enrichment step comprises an affinity chromatography step, in some cases preceded by a step of elimination of cells and/or cell debris (clarification). A particular enrichment step according to this disclosure comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 μm, e.g. between 0.2 and 10 μm, may be used. It is particularly possible to use a succession of filters with a porosity of 10 μm, followed by 1 μm, 0.5 μm, and then 0.22 μm.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to an embodiment, the biological sample (e.g., the supernatant) is subjected to an ultrafiltration, such as a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the disclosure, the use of membranes with a cut-off threshold below 1000 kDa, such as between 300 kDa and 1000 kDa, for example between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. In some embodiments, an affinity chromatography on a dye is used, allowing the elimination (i.e., the retention) of contaminants such as proteins and enzymes, for instance albumin, kinases, dehydrogenases, clotting factors, interferons, lipoproteins, or also co-factors, etc. In some embodiments, the support used for this chromatography step is a support as used for the ion exchange chromatography, functionalized with a dye. As specific example, the dye may be selected from Blue SEPHAROSE™ (Pharmacia), YELLOW 86, GREEN 5, and BROWN 10 (Sigma). The support may be agarose. It should be understood that any other support and/or dye or reactive group allowing the retention (binding) of contaminants from the treated biological sample can be used in the present disclosure.

### V. Administration of Therapeutic Compositions

The therapy provided herein may comprise administration of a therapeutic agents (e.g., fibroblasts, exosomes from fibroblasts, fibroblast-derived products, etc.) alone or in combination. Therapies may be administered in any suitable manner known in the art. For example, a first and second treatment (e.g., fibroblast-derived products and diphenylcyclopropenone) may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts, fibroblast-derived products, diphenylcyclopropenone) of the disclosure may be administered by the same route of administration or by different routes of administration. In some embodiments, the therapeutic agent is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Use of Conditioned Media from Fibroblasts for Treatment of Androgenetic Alopecia

Preparation of exosomes: Fibroblasts are cultured under conditions sufficient to promote release of exosomes into the media. Specifically, foreskin fibroblasts are cultured in 15 ml alpha MEM-media containing 10% fetal calf serum in T 175 flasks. After a 24 hour culture, media is replaced with phosphate buffered saline and cells are cultured for an additional 12 hours. Exosomes are extracted by gradient centrifugation, specifically 10 min at 300 g, 10 min at 2000 g, 30 min at 10000 g, followed by exosome pelleting by centrifugation at 100000 g for 70 min. A repeated 100000 g centrifugation of the re-suspended pellet is optionally applied to purify the exosome preparation from the lower mobility fractions, mainly from free proteins.

Clinical Trial for Treatment of Androgenetic alopecia (AGA): Thirty male patients that met the eligibility criteria and provided written informed consent are enrolled. A dermatologist makes the clinical diagnosis of AGA in all participants. The inclusion criteria are age of 18-60 years, hair loss longer than 6 months, and Hamilton-Norwood grade of 3-6. Those with scalp infection, malignant disease, autoimmune diseases (Hashimoto, rheumatoid arthritis, lupus, etc.), history of vascular surgery in the past 3 months, any systemic disease interacting with hair growth, telogen effluvium, history of hematologic disorders, and addiction to oral or intravenous narcotics are excluded from study.

Other exclusion criteria are consumption of topical or systemic treatment for hair loss in the past 6 months, receiving chemotherapy or any immunosuppressive medication, use of any growth factors containing medications, hemoglobin level below 10, thrombocytopenia (or platelet count below 100,000), serum albumin below 2.5 g/dl, and participation in another clinical trial (within the last 3 months).

15 patients are treated with 1 microgram of exosome protein per ml every 2 weeks for 5 sessions. The solution is injected intradermally in the temporal and frontal areas, 0.05 ml per area in 1-2 cm intervals. 15 additional patients are administered fibroblast cells with the same frequency at a concentration of 5 million fibroblasts per scalp.

At the beginning of the study before the intervention and 3 months after the last treatment session standard photographs are taken with digital cameras (Nikon D300s®, Tokyo, Japan), trichogram is performed, and hair density and diameter is measured using the digital photographic hair analyzer (KC Triple Scope®, KC Technology Co, Korea) by a 150× lens on an area marked with a tattoo. In addition, patients complete a patient satisfaction questionnaire at each visit on a −2 to +2 score (−2: much worse, −1: slightly worse, 0: without change, +1: slightly better, +2: much better).

Percentage and frequency are used to describe qualitative data, and mean and standard deviation are used for description of quantitative data. Comparison of quantitative data before and after the test is performed by paired t-test (or nonparametric equivalent, depending on data distribution or categorized information). Estimation of all tests is done on significance level of 5%.

All parameters significantly improved after 5 weeks of treatment.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications, including those listed below, are herein incorporated by reference to the same extent as if each individual publication was specifically and individually indicated to be incorporated by reference.
1. Askin, O., Z. Koyuncu, and S. Serdaroglu, Association of alopecia with self-esteem in children and adolescents. Int J Adolesc Med Health, 2020.
2. MacLean, K. J. and M. J. Tidman, Alopecia areata: more than skin deep. Practitioner, 2013. 257(1764): p. 29-32, 3.
3. Dai, Y. X., et al., Cigarette Smoking, Alcohol Consumption, and Risk of Alopecia Areata: A Population-Based Cohort Study in Taiwan. Am J Clin Dermatol, 2020. 21(6): p. 901-911.
4. Paus, R., et al., Pathobiology of chemotherapy-induced hair loss. Lancet Oncol, 2013. 14(2): p. e50-9.
5. Peters, E. M., et al., A new strategy for modulating chemotherapy-induced alopecia, using PTH/PTHrP receptor agonist and antagonist. J Invest Dermatol, 2001. 117(2): p. 173-8.
6. Paik, S. H., et al., Pretreatment of epidermal growth factor promotes primary hair recovery via the dystrophic anagen pathway after chemotherapy-induced alopecia. Exp Dermatol, 2013. 22(7): p. 496-9.
7. Yoon, J. S., et al., Development of a Model for Chemotherapy-Induced Alopecia: Profiling of Histological Changes in Human Hair Follicles after Chemotherapy. J Invest Dermatol, 2016. 136(3): p. 584-92.
8. Lv, S., et al., A Case of Acute Telogen Effluvium After SARS-CoV-2 Infection. Clin Cosmet Investig Dermatol, 2021. 14: p. 385-387.
9. Sattar, F., et al., Efficacy of Oral Vitamin D3 Therapy in Patients Suffering from Diffuse Hair Loss (Telogen Effluvium). J Nutr Sci Vitaminol (Tokyo), 2021. 67(1): p. 68-71.
10. Cheng, T., et al., The Diagnostic Value of Serum Ferritin for Telogen Effluvium: A Cross-Sectional Comparative Study. Clin Cosmet Investig Dermatol, 2021. 14: p. 137-141.
11. Chien Yin, G. O., J. L. Siong-See, and E. C. E. Wang, Telogen Effluvium—a review of the science and current obstacles. J Dermatol Sci, 2021. 101(3): p. 156-163.
12. Olds, H., et al., Telogen effluvium associated with COVID-19 infection. Dermatol Ther, 2021. 34(2): p. e14761.
13. Kakpovbia, E., et al., Laboratory Testing in Telogen Effluvium. J Drugs Dermatol, 2021. 20(1): p. 110-111.
14. Trueb, R. M., H. Dutra Rezende, and M. F. R. Gavazzoni Dias, What can the hair tell us about COVID-19? Exp Dermatol, 2021. 30(2): p. 288-290.
15. Cline, A., et al., A surge in the incidence of telogen effluvium in minority predominant communities heavily impacted by COVID-19. J Am Acad Dermatol, 2021. 84(3): p. 773-775.
16. Rizzetto, G., et al., Telogen effluvium related to post severe Sars-Cov-2 infection: Clinical aspects and our management experience. Dermatol Ther, 2021. 34(1): p. e14547.
17. Hughes, E. C. and D. Saleh, Telogen Effluvium, in StatPearls. 2021: Treasure Island (FL).
18. Kageyama, R., et al., Immunological Properties of Atopic Dermatitis-Associated Alopecia Areata. Int J Mol Sci, 2021. 22(5).
19. Malviya, N., et al., Cutaneous immune-related adverse events to checkpoint inhibitors. Clin Dermatol, 2020. 38(6): p. 660-678.
20. Zito, P. M. and B. S. Raggio, Hair Transplantation, in StatPearls. 2021: Treasure Island (FL).
21. Shome, D., et al., “Comparison of QR 678((R)) & QR678((R)) Neo as monotherapy and as combination therapy with 5% Minoxidil solution and oral Finasteride in the treatment of male androgenetic alopecia—Which is better?”. J Cosmet Dermatol, 2021.
22. Jha, A. K., et al., Platelet-rich plasma with low dose oral minoxidil (1.25 mg versus 2.5 mg) along with trichoscopic pre- and post-treatment evaluation. J Cosmet Dermatol, 2021.
23. Villani, A., et al., Review of oral minoxidil as treatment of hair disorders: in search of the perfect dose. J Eur Acad Dermatol Venereol, 2021.
24. Zhao, J., et al., Treatment of alopecia by transplantation of hair follicle stem cells and dermal papilla cells encapsulated in alginate gels. Med Hypotheses, 2008. 70(5): p. 1014-6.
25. Mahasaksiri, T., et al., Application of Topical Immunotherapy in the Treatment of Alopecia Areata: A Review and Update. Drug Des Devel Ther, 2021. 15: p. 1285-1298.
26. Gong, Y., et al., Diphenylcyclopropenone plays an effective therapeutic role by up-regulating the TSLP/OX40L/IL-13 pathway in severe alopecia areata. Exp Dermatol, 2021. 30(2): p. 278-283.
27. Abedini, R., et al., Utility of Dermoscopic Evaluation in Predicting Clinical Response to Diphencyprone in a Cohort of Patients with Alopecia Areata. Int J Trichology, 2020. 12(3): p. 107-113.
28. Kagami, S., Y. Kishi, and H. Hino, Topical immunotherapy in combination with anthralin in the treatment of refractory alopecia areata. J Cosmet Dermatol, 2020. 19(9): p. 2411-2414.
29. Nasimi, M., et al., Topical immunotherapy with diphenylcyclopropenone in patients with alopecia areata: A large retrospective study of 757 patients. Dermatol Ther, 2020. 33(6): p. e13808.
30. Gupta, A. K., et al., Monotherapy for Alopecia Areata: A Systematic Review and Network Meta-Analysis. Skin Appendage Disord, 2019. 5(6): p. 331-337.
31. Kim, B. J., et al., Home-based contact immunotherapy with diphenylcyclopropenone improves compliance with the recommended follow-up for patients with alopecia areata: A retrospective cohort study. J Am Acad Dermatol, 2020. 82(5): p. 1223-1225.
32. Kutlubay, Z., et al., Assessment of treatment efficacy of diphenylcyclopropenone (DPCP) for alopecia areata. Turk J Med Sci, 2020. 50(8): p. 1817-1824.
33. Herz-Ruelas, M. E., et al., Immunotherapy for resistant and/or severe alopecia areata in a university hospital setting in Northern Mexico. Australas J Dermatol, 2020. 61(1): p. e137-e139.
34. Ibrahim, S. A., A. M. Esawy, and A. S. Abdelshafy, Treatment of chronic extensive alopecia areata by diphenylcyclopropenone alone versus in combination with anthralin. Dermatol Ther, 2019. 32(5): p. e13010.
35. Nowicka, D., et al., Efficacy of diphenylcyclopropenone in alopecia areata: a comparison of two treatment regimens. Postepy Dermatol Alergol, 2018. 35(6): p. 577-581.
36. Lee, S., et al., Hair Regrowth Outcomes of Contact Immunotherapy for Patients With Alopecia Areata: A Systematic Review and Meta-analysis. JAMA Dermatol, 2018. 154(10): p. 1145-1151.
37. Gong, Y., et al., Serum level of IL-4 predicts response to topical immunotherapy with diphenylcyclopropenone in alopecia areata. Exp Dermatol, 2020. 29(3): p. 231-238.
38. Lee, S. H., et al., Can the Cytokine Analysis of the Scales on Alopecic Patch Predict the Response to Diphenylcyclopropenone Treatment in Alopecia Areata Patients? Ann Dermatol, 2018. 30(2): p. 150-157.
39. Sriphojanart, T., S. Khunkhet, and P. Suchonwanit, A retrospective comparative study of the efficacy and safety of two regimens of diphenylcyclopropenone in the treatment of recalcitrant alopecia areata. Dermatol Reports, 2017. 9(2): p. 7399.
40. Choe, S. J., et al., Subclinical sensitization with diphenylcyclopropenone is sufficient for the treatment of alopecia areata: Retrospective analysis of 159 cases. J Am Acad Dermatol, 2018. 78(3): p. 515-521 e4.
41. Estefan, J., et al., Alopecia Areata-Part III: Prognosis and Treatment. Skinmed, 2016. 14(5): p. 361-365.
42. Hoffmann, R., et al., Cytokine mRNA levels in Alopecia areata before and after treatment with the contact allergen diphenylcyclopropenone. J Invest Dermatol, 1994. 103(4): p. 530-3.
43. Chen, G., et al., Exosomal PD-L1 contributes to immunosuppression and is associated with anti-PD-1 response. Nature, 2018. 560(7718): p. 382-386.
44. Savci, U., et al., Ischemia-modified albumin as a possible marker of oxidative stress in patients with telogen effluvium. An Bras Dermatol, 2020. 95(4): p. 447-451.
45. Flores, A., et al., Inhibition of pyruvate oxidation as a versatile stimulator of the hair cycle in models of alopecia. Exp Dermatol, 2021. 30(4): p. 448-456.
46. Abbas, A. N., Ginger (Zingiber officinale (L.) Rosc) improves oxidative stress and trace elements status in patients with alopecia areata. Niger J Clin Pract, 2020. 23(11): p. 1555-1560.
47. Guo, Y., et al., Proposed mechanisms of low-level light therapy in the treatment of androgenetic alopecia. Lasers Med Sci, 2020.
48. Mustafa, A. I., et al., Cross talk between oxidative stress and inflammation in alopecia areata. J Cosmet Dermatol, 2020.
49. Balik, A. R., et al., Examination of androgenetic alopecia with serum biomarkers. J Cosmet Dermatol, 2020.
50. Cakirca, G., et al., Effects of anxiety and depression symptoms on oxidative stress in patients with alopecia areata. Postepy Dermatol Alergol, 2020. 37(3): p. 412-416.
51. Cwynar, A., D. M. Olszewska-Slonina, and R. Czajkowski, The impact of oxidative stress in androgenic alopecia in women. Postepy Dermatol Alergol, 2020. 37(1): p. 119-120.
52. Acharya, P. and M. C. Mathur, Oxidative stress in alopecia areata: a systematic review and meta-analysis. Int J Dermatol, 2020. 59(4): p. 434-440.
53. Chen, Q., et al., Hydroxytyrosol prevents dermal papilla cells inflammation under oxidative stress by inducing autophagy. J Biochem Mol Toxicol, 2019. 33(9): p. e22377.
54. Cwynar, A., et al., Evaluation of selected parameters of oxidative stress in patients with alopecia areata. Postepy Dermatol Alergol, 2019. 36(1): p. 115-116.
55. Lim, Y. C., et al., Genetic analysis of a novel antioxidant multi-target iron chelator, M30 protecting against chemotherapy-induced alopecia in mice. BMC Cancer, 2019. 19(1): p. 149.
56. Incel-Uysal, P., et al., Assessment of Metabolic Profile and Ischemia-modified Albumin Level in Patients with Alopecia Areata: A Case-Control Study. Indian J Dermatol, 2019. 64(1): p. 12-18.
57. Cwynar, A., et al., Investigation of oxidative stress in patients with alopecia areata by measuring the levels of malondialdehyde and ceruloplasmin in the blood. Postepy Dermatol Alergol, 2018. 35(6): p. 572-576.
58. Dizen-Namdar, N., et al., Evaluation of Serum Paraoxonase, Arylesterase, Prolidase Activities and Oxidative Stress in Patients with Alopecia Areata. Skin Pharmacol Physiol, 2019. 32(2): p. 59-64.
59. Riccio, G., et al., Annurca Apple Polyphenols Protect Murine Hair Follicles from Taxane Induced Dystrophy and Hijacks Polyunsaturated Fatty Acid Metabolism toward beta-Oxidation. Nutrients, 2018. 10(11).
60. Badolati, N., et al., Annurca Apple Polyphenols Ignite Keratin Production in Hair Follicles by Inhibiting the Pentose Phosphate Pathway and Amino Acid Oxidation. Nutrients, 2018. 10(10).
61. Maekawa, M., et al., Thiosulfate promotes hair growth in mouse model. Biosci Biotechnol Biochem, 2019. 83(1): p. 114-122.
62. Singh, B., et al., Reversing wrinkled skin and hair loss in mice by restoring mitochondrial function. Cell Death Dis, 2018. 9(7): p. 735.
63. Hatem, S., et al., Melatonin vitamin C-based nanovesicles for treatment of androgenic alopecia: Design, characterization and clinical appraisal. Eur J Pharm Sci, 2018. 122: p. 246-253.
64. Jadkauskaite, L., et al., Oxidative stress management in the hair follicle: Could targeting NRF2 counter age-related hair disorders and beyond? Bioessays, 2017. 39(8).
65. Kaya Erdogan, H., et al., The role of oxidative stress in early-onset androgenetic alopecia. J Cosmet Dermatol, 2017. 16(4): p. 527-530.
66. Prie, B. E., et al., Oxidative stress in androgenetic alopecia. J Med Life, 2016. 9(1): p. 79-83.
67. Nicu, C., et al., Dermal adipose tissue secretes HGF to promote human hair growth and pigmentation. J Invest Dermatol, 2021.
68. Sano, S., et al., Tissue regeneration: hair follicle as a model. J Investig Dermatol Symp Proc, 2001. 6(1): p. 43-8.
69. Otomo, S., [Hair growth effect of minoxidil]. Nihon Yakurigaku Zasshi, 2002. 119(3): p. 167-74.
70. Zhou, Y., et al., Autologous activated platelet-rich plasma in hair growth: A pilot study in male androgenetic alopecia with in vitro bioactivity investigation. J Cosmet Dermatol, 2021. 20(4): p. 1221-1230.

What is claimed is:

1. A method of treating or preventing alopecia in a subject comprising providing to the subject an effective amount of fibroblasts or fibroblast-derived products.

2. The method of claim 1, wherein the alopecia is associated with dermal inflammation.

3. The method of claim 1 or 2, wherein the alopecia is alopecia areata.

4. The method of claim 1, wherein the alopecia is androgenetic alopecia.

5. The method of claim 4, wherein the androgenetic alopecia is male-pattern hair loss.

6. The method of claim 4, wherein the androgenetic alopecia is female-pattern hair loss.

7. The method of any of claims 1-6, wherein the fibroblasts or fibroblast-derived products are administered topically.

8. The method of any of claims 1-6, wherein the fibroblasts or fibroblast-derived products are administered intradermally and/or transdermally.

9. The method of any of claims 1-8, wherein the fibroblasts or fibroblast-derived products are administered to a region of the scalp of the subject.

10. The method of claim 9, further comprising administering a regenerative light source to the region of the scalp of the subject.

11. The method of any of claims 1-10, wherein the method comprises providing to the subject an effective amount of fibroblasts.

12. The method of claim 11, wherein the fibroblasts are allogeneic, xenogeneic, or autologous to the subject.

13. The method of claim 11 or 12, wherein the fibroblasts are derived from skin, adipose, bone marrow, omental tissue, blood, deciduous teeth, fallopian tubes, testicular tissue, ovarian tissue, hair follicle, endometrial tissue, or a combination thereof.

14. The method of any of claims 11-13, wherein the fibroblasts are dermal fibroblasts.

15. The method of any of claims 11-14, wherein the fibroblasts were previously subjected to conditions sufficient to enhance regenerative activity.

16. The method of claim 15, wherein the conditions are sufficient to upregulate HIF1α expression in the fibroblasts.

17. The method of claim 16, wherein the conditions are sufficient to upregulate HIF1α by at least 25% relative to an untreated control.

18. The method of any of claims 11-14, wherein the conditions are sufficient to enhance nuclear translocation of HIF1α in the fibroblasts.

19. The method of any of claims 11-18, wherein the conditions comprise agents capable of imitating hypoxia.

20. The method of any of claims 11-19, wherein the conditions comprise culturing the fibroblasts with carbon monoxide.

21. The method of claim 20, wherein the conditions comprise exposing the fibroblasts to a gas composition comprising between about 0% and about 79% nitrogen by weight, between about 21% and about 99.999999% oxygen by weight, and between 0.0000001% and about 0.3% carbon monoxide by weight.

22. The method of claim 21, wherein the gas composition comprises 0% nitrogen and about 99.999999% oxygen.

23. The method of any of claims 20-22, wherein the gas composition comprises between about 0.005% and about 0.05%.

24. The method of any of claims 11-23, wherein the fibroblasts were previously subjected to conditions sufficient to enhance survival and/or activity of the fibroblasts.

25. The method of claim 24, wherein the conditions comprise treatment with an epigenetic modulator.

26. The method of claim 25, wherein the epigenetic modulator is a histone deacetylase inhibitor.

27. The method of claim 26, wherein the histone deacetylase inhibitor is valproic acid, vorinostat, entinostat, panobinostat, trichostatin A, mocetinostat, belinostat, FK228, MC1568, tubastatin, sodium butyrate, or sulforaphane.

28. The method of claim 25, wherein the epigenetic modulator is a DNA methyltransferase inhibitor.

29. The method of claim 28, wherein the DNA methyltransferase inhibitor is 5-azacytidine.

30. The method of claim 24, wherein the conditions comprise culturing the fibroblasts with GSK-3 inhibitor.

31. The method of claim 30, wherein the GSK-3 inhibitor is lithium or a lithium salt.

32. The method of any of claims 1-10, wherein the method comprises providing to the subject an effective amount of fibroblast-derived products.

33. The method of claim 32, wherein the fibroblast-derived products were obtained from fibroblasts derived from skin, adipose, bone marrow, omental tissue, blood, deciduous teeth, fallopian tubes, testicular tissue, ovarian tissue, hair follicle, endometrial tissue, or a combination thereof.

34. The method of claim 33, wherein the fibroblast-derived products were obtained from dermal fibroblasts.

35. The method of any of claims 32-34, wherein the fibroblast-derived products were obtained from fibroblasts that are allogeneic, xenogeneic, or autologous to the subject.

36. The method of any of claims 32-35, wherein the fibroblast-derived products comprise conditioned media from culture of fibroblasts.

37. The method of claim 36, wherein the conditioned media was obtained from culture of the fibroblasts in EMEM, alpha-MEM, IMDM, DMEM, or RPMI.

38. The method of claim 36 or 37, wherein the conditioned media was generated by culture of adherent fibroblasts in a liquid suspension comprising nutrition for the fibroblasts.

39. The method of claim 38, wherein the liquid suspension comprises a growth factor.

40. The method of claim 38 or 39, wherein the liquid suspension comprises stem cell exosomes.

41. The method of claim 40, wherein the stem cell exosomes are exosomes from mesenchymal stem cells.

42. The method of claim 41, wherein the mesenchymal stem cells were derived from umbilical cord, bone marrow, skin, fallopian tube, adipose tissue, endometrial tissue, peripheral blood, menstrual blood, hair follicle, or a combination thereof.

43. The method of any of claims 38-42, wherein the liquid suspension comprises a neutralizing factor capable of inhibiting activity of one or more inflammatory mediators.

44. The method of claim 43, wherein the neutralizing factor is a monoclonal antibody, an antisense oligonucleotide, or a gene editing system.

45. The method of claim 43 or 44, wherein the neutralizing factor is an antibody capable of binding to interleukin-1, interleukin-6, interleukin-8, interleukin-9, interleukin-11, interleukin-12, interleukin-15, interleukin-17, interleukin-18, interleukin-21, interleukin-23, interleukin-27, interleukin-33, TNFα, interferon gamma, TNFβ, or lymphotoxin.

46. The method of any of claims 38-45, wherein the liquid suspension comprises VEGF, EGF, PGDF-BB, IGF-1, HGF-1, NGF, BDNF, IL-3, IL-4, IL-10, IL-13, IL-20, IL-35.

47. The method of any of claims 32-35, wherein the fibroblast-derived products are microvesicles from fibroblasts.

48. The method of any of claims 32-35, wherein the fibroblast-derived products are exosomes from fibroblasts.

49. The method of claim 48, wherein the exosomes were concentrated from conditioned media from the fibroblasts.

50. The method of claim 49, wherein the exosomes were concentrated by a) functionalizing a support with a single stranded oligonucleotide to generate a functionalized support; b) incubating the functionalized support with a ligand having a tag which is complimentary to the single strand oligonucleotide order to obtain an immobilized ligand; c) incubating the immobilized ligand with the conditioned media to allow the capture of the exosomes through the binding of said immobilized ligand with the exosomes to obtain a substrate of captured exosomes; and d) incubating the captured exosomes with a restriction enzyme.

51. The method of claim 50, wherein the ligand is an antibody, a peptide, or an aptamer.

52. The method of claim 50 or 51, wherein the support is a magnetic bead, a membrane, a cell culture plate, a test tube, a slide, a microplate, a microchannel, a pillar, or a disk-like piece.

53. The method of any of claims 50-52, wherein the support is functionalized with the ligand by covalent bonding or via biotinylation.

54. The method of any of claims 50-53, wherein the restriction enzyme is a DNAse.

55. The method of any of claims 50-54, wherein the ligand is an antibody capable of binding to an exosome-specific tetraspanin.

56. The method of any of claims 50-54, wherein the ligand is an antibody capable of binding to MHC class I and II, HSP70, Annexin V, Flotillin, or EpCAM.

57. The method of any of claims 49-56, wherein the conditioned media is derived from culture of the fibroblasts in EMEM, alpha-MEM, IMDM, DMEM, or RPMI.

58. The method of any of claims 32-35, wherein the fibroblast-derived products are apoptotic vesicles from fibroblasts.

59. The method of any of claims 32-35, wherein the fibroblast-derived products are nucleic acids from fibroblasts.

60. The method of any of claims 32-59, wherein the fibroblast-derived products were obtained from fibroblasts subjected to conditions sufficient to enhance regenerative activity.

61. The method of claim 60, wherein the conditions are sufficient to upregulate HIF1α expression in the fibroblasts.

62. The method of claim 61, wherein the conditions are sufficient to upregulate HIF1α by at least 25% relative to an untreated control.

63. The method of claim 60, wherein the conditions are sufficient to enhance nuclear translocation of HIF1α in the fibroblasts.

64. The method of claim 60, wherein the conditions comprise agents capable of imitating hypoxia.

65. The method of claim 60, wherein the conditions comprise culturing the fibroblasts with carbon monoxide.

66. The method of claim 65, wherein the conditions comprise exposing the fibroblasts to a gas composition comprising between about 0% and about 79% nitrogen by weight, between about 21% and about 99.999999% oxygen by weight, and between 0.0000001% and about 0.3% carbon monoxide by weight.

67. The method of claim 66, wherein the gas composition comprises 0% nitrogen and about 99.999999% oxygen.

68. The method of claim 66 or 67, wherein the gas composition comprises between about 0.005% and about 0.05%.

69. The method of any of claims 32-59, wherein the fibroblast-derived products were obtained from fibroblasts subjected to conditions sufficient to enhance survival and/or activity of the fibroblasts.

70. The method of claim 69, wherein the conditions comprise treatment with an epigenetic modulator.

71. The method of claim 70, wherein the epigenetic modulator is a histone deacetylase inhibitor.

72. The method of claim 71, wherein the histone deacetylase inhibitor is valproic acid, vorinostat, entinostat, panobinostat, trichostatin A, mocetinostat, belinostat, FK228, MC1568, tubastatin, sodium butyrate, or sulforaphane.

73. The method of claim 70, wherein the epigenetic modulator is a DNA methyltransferase inhibitor.

74. The method of claim 73, wherein the DNA methyltransferase inhibitor is 5-azacytidine.

75. The method of claim 69, wherein the conditions comprise culturing the fibroblasts with GSK-3 inhibitor.

76. The method of claim 75, wherein the GSK-3 inhibitor is lithium or a lithium salt.

77. The method of any of claims 1-76, wherein, prior to providing the fibroblasts or fibroblast-derived products, the subject has a reduced number of cells expressing FoxP3 compared with an age matched control subject.

78. The method of any of claims 1-76, wherein, prior to providing the fibroblasts or fibroblast-derived products, the subject has a reduced number of cells expressing interleukin-10 compared with an age matched control subject.

79. The method of any of claims 1-76, wherein, prior to providing the fibroblasts or fibroblast-derived products, the subject has a reduced number of cells expressing interleukin-4 compared with an age matched control subject.

80. The method of any of claims 1-76, wherein, prior to providing the fibroblasts or fibroblast-derived products, the subject has a reduced number of cells expressing interleukin-13 compared with an age matched control subject.

81. The method of any of claims 1-76, wherein, prior to providing the fibroblasts or fibroblast-derived products, the subject has a reduced number of cells expressing interleukin-35 compared with an age matched control subject.

82. The method of any of claims 1-76, wherein, prior to providing the fibroblasts or fibroblast-derived products, the subject has a reduced number of T regulatory cells compared with an age matched control subject.

83. The method of any of claims 1-76, wherein, prior to providing the fibroblasts or fibroblast-derived products, the subject has a reduced number of myeloid suppressor cells compared with an age matched control subject.

84. The method of any of claims 1-76, wherein, prior to providing the fibroblasts or fibroblast-derived products, the subject has a reduced number of B cells expressing TIM-1 compared with an age matched control subject.

85. The method of any of claims 1-76, wherein, prior to providing the fibroblasts or fibroblast-derived products, the subject has a reduced number of B cells expressing IL-10 compared with an age matched control subject.

86. The method of any of claims 1-76, wherein, prior to providing the fibroblasts or fibroblast-derived products, the subject has a reduced number of B regulatory cells compared with an age matched control subject.

87. The method of any of claims 1-76, wherein, prior to providing the fibroblasts or fibroblast-derived products, the subject has an increased number of cells expressing interferon gamma compared with an age matched control subject.

88. The method of any of claims 1-76, wherein, prior to providing the fibroblasts or fibroblast-derived products, the subject has an increased number of cells expressing TNF-alpha compared with an age matched control subject.

89. The method of any of claims 1-76, wherein, prior to providing the fibroblasts or fibroblast-derived products, the subject has an increased number of cells expressing interleukin-1 compared with an age matched control subject.

90. The method of any of claims 1-76, wherein, prior to providing the fibroblasts or fibroblast-derived products, the subject has an increased number of cells expressing interleukin-2 compared with an age matched control subject.

91. The method of any of claims 1-76, wherein, prior to providing the fibroblasts or fibroblast-derived products, the subject has an increased number of cells expressing interleukin-6 compared with an age matched control subject.

92. The method of any of claims 1-76, wherein, prior to providing the fibroblasts or fibroblast-derived products, the subject has an increased number of cells expressing interleukin-8 compared with an age matched control subject.

93. The method of any of claims 1-76, wherein, prior to providing the fibroblasts or fibroblast-derived products, the subject has an increased number of cells expressing interleukin-11 compared with an age matched control subject.

94. The method of any of claims 1-76, wherein, prior to providing the fibroblasts or fibroblast-derived products, the subject has an increased number of cells expressing interleukin-12 compared with an age matched control subject.

95. The method of any of claims 1-76, wherein, prior to providing the fibroblasts or fibroblast-derived products, the subject has an increased number of cells expressing interleukin-15 compared with an age matched control subject.

96. The method of any of claims 1-76, wherein, prior to providing the fibroblasts or fibroblast-derived products, the subject has an increased number of cells expressing interleukin-17 compared with an age matched control subject.

97. The method of any of claims 1-76, wherein, prior to providing the fibroblasts or fibroblast-derived products, the subject has an increased number of cells expressing interleukin-18 compared with an age matched control subject.

98. The method of any of claims 1-76, wherein, prior to providing the fibroblasts or fibroblast-derived products, the subject has an increased number of cells expressing interleukin-21 compared with an age matched control subject.

99. The method of any of claims 1-76, wherein, prior to providing the fibroblasts or fibroblast-derived products, the subject has an increased number of cells expressing interleukin-23 compared with an age matched control subject.

100. The method of any of claims 1-76, wherein, prior to providing the fibroblasts or fibroblast-derived products, the subject has an increased number of cells expressing interleukin-27 compared with an age matched control subject.

101. The method of any of claims 1-76, wherein, prior to providing the fibroblasts or fibroblast-derived products, the subject has an increased number of cells expressing interleukin-33 compared with an age matched control subject.

102. The method of any of claims 1-76, wherein, prior to providing the fibroblasts or fibroblast-derived products, the subject has an increased number of natural killer cells compared with an age matched control subject.

103. The method of any of claims 1-76, wherein, prior to providing the fibroblasts or fibroblast-derived products, the subject has an increased number of natural killer T cells compared with an age matched control subject.

104. The method of any of claims 1-76, wherein, prior to providing the fibroblasts or fibroblast-derived products, the subject has an increased number of Th1 cells compared with an age matched control subject.

105. The method of any of claims 1-76, wherein, prior to providing the fibroblasts or fibroblast-derived products, the subject has an increased number of Th17 cells compared with an age matched control subject.

106. The method of any of claims 1-105, further comprising providing to the subject an effective amount of diphenylcyclopropenone.

107. A method of treating or preventing alopecia areata in a subject comprising providing to the subject an effective amount of fibroblast-derived exosomes.

108. A method of treating or preventing alopecia areata in a subject comprising providing to the subject an effective amount of conditioned media from fibroblasts.

109. The method of claim 107 or 108, further comprising providing to the subject an effective amount of diphenylcyclopropenone.

110. A method of treating or preventing androgenetic alopecia in a subject comprising providing to the subject an effective amount of fibroblast-derived exosomes.

111. A method of treating or preventing androgenetic alopecia in a subject comprising providing to the subject an effective amount of conditioned media from fibroblasts.
